(19) United States
(12) Patent Application Publication
(10) Pub. No.: US 2006/0122619 A1
(43) Pub. Date: Jun. 8, 2006

(54) Devices and systems for illuminating or irradiating a light-sensitive sealant for polymerization and cross-linking and methods of using the same

(76) Inventors: Joseph J. Kablik, Tyngsborough, MA (US); Michael Dumont, Hampton, NH (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 11/004,592

(22) Filed: Dec. 3, 2004

(51) Int. Cl. A61B 17/58; A61F 2/38

(52) U.S. Cl. 606/88

(57) ABSTRACT

Disposable, self-contained, hand-held, low power devices for irradiating or illuminating a light sensitive material, such as a sealant, hydrogel, and the like, that has been applied to soft, living mammalian tissue and systems and methods using the devices are disclosed. Preferably, the light sources are low power light emitting diodes that provide a desired power density for a predetermined period of time that are controlled by a control board that limits the current and the time of application of the activating energy. The illumination or irradiation causes the light sensitive material to cure, polymerize, bond, and/or cross-link with the tissue to provide a suitable, sealant (modulus) strength.

### BACKGROUND OF THE INVENTION

1. Field of the Invention

The present invention relates to devices, systems, and methods for applying an initially fluent material to the surface of mammalian tissue and polymerizing the material to a non-fluent state. More specifically, the present invention relates to devices, systems, and methods for applying an initially fluent light-sensitive material to soft, living tissue and activating the material using a light source so that the material cures, polymerizes, bonds, and/or cross-links with the tissue.

2. Background Art

The application of polymeric materials, e.g., sealants, hydrogels, and the like, to body tissues of human or animal patients, i.e., mammalian patients, is becoming increasingly important in medicine. For example, sealants, hydrogels, and the like can be applied to living tissue to prevent post-operative adhesions; to protect tissue surfaces; to alter the tissue; to create or preserve lumens, channels or reservoirs for the passage or collection of fluids; to create matrices for the growth of tissue; to control undesirable tissue growth; to deliver therapeutic agents to a tissue surface; to join a tissue surface to other tissue(s) or an artificial implant; to isolate or protect tissue or lesions to enable or mediate healing; to mediate the rate of substances or energy passing into, out of, or through the tissue; for the local application of biologically active species, and for the controlled release of biologically active agents to achieve local and systemic effects. Sealants, hydrogels, and the like, further, can be used as temporary or long-term tissue adhesives or as materials for filling voids in biological materials. The materials and conditions of application are selected to enhance desirable properties such as good tissue adherence without adverse tissue reaction, non-toxicity, good biocompatibility, biodegradability when desired, and ease of application or handling.

Typically, the composition that will form the polymerized sealants, hydrogels, and the like can include a light sensitive polymerization initiator that is applied to the tissue surface in fluent form, e.g., as a liquid. The coated tissue then is exposed to light in situ to cure, or polymerize, the composition, rendering it non-fluent. Conventionally, the illuminating or irradiating light is selected to have an appropriate wavelength to initiate or sustain the polymerization efficiently and, moreover, to have an appropriate light intensity to achieve polymerization within the desired time.

Cross-linking is achieved by irradiating, or illuminating, the article with light of a wavelength or within a wavelength range at which the polymeric material readily absorbs. Necessarily, this is achieved by providing polymeric material that absorbs the radiation provided. The cross-linked sealant, hydrogel and the like provides a chemical or mechanical bond to the tissue.

Although it has been recognized that the use of polymeric materials in vivo may offer significant therapeutic effects, such applications have met many limitations. For example, the methods for applying such polymers to tissue surfaces often require the use of pressure, heat or electrical energy exceeding tolerable limits at the tissue site. Likewise various chemical effects associated with such polymers have been found to be physiologically unacceptable.

The viscosity of non-photo-polymeized fluent sealants, hydrogels, and the like is another source of problems with the prior art. Less viscous fluids can more easily run off of the tissue, making curing more difficult. Conversely, more viscous fluids do not flow off the tissue as readily, requiring greater cure time for the material to flow over the application surface. As a result, controlling the chemical curing process is one of give and take.

Numerous methods for reshaping polymeric materials in vivo are known in the prior art. For example, U.S. Pat. No. 5,213,580 and International Publication WO 90/01969, both to Slepian et al., which are incorporated herein by reference, describe methods in which polymers having melting points slightly above physiological temperatures are implanted into a patient. The polymers are melted via contact with heated fluids and shaped using mechanical force provided by, e.g., a balloon catheter. Unfortunately, many of the methods known in the art suffer from the need to use energy levels beyond those that are physiologically tolerable, or from the inability to sufficiently control the shape change and/or temperature of the polymeric material.

Typically, the primary limitation in prior art methods for the delivery of energy to an implanted material or device is the inability to direct the energy specifically to the material or device, while minimizing energy delivery to adjacent or neighboring body tissue. For example, it is known in the prior art that polymeric devices such as stents can be delivered to specific locations in vivo, e.g., using a balloon catheter. Such stents can be heated at the site, e.g., by filling the balloon portion with a heated fluid. Preferably, heat is conducted from the fluid in the balloon portion, through the balloon material, and into the stent. Since conduction is a relatively slow process and the balloon portion has a relative large thermal mass, energy is transferred to the surrounding body tissues and fluids as well as to the stent. As a result, undesired amounts of heat are transferred into the surrounding body tissues and fluids.

U.S. Pat. No. 5,779,673 to Roth, et al., the teachings of which also are incorporated herein by reference, provides apparatuses and methods for delivering polymeric material in vivo, and more particularly to the implantation of polymeric material into tissue lumens of mammalian patients. Further, the Roth patent provides methods for photo-thermoforming a polymeric article in vivo, which is to say, modifying the shape of a polymeric article in vivo by selectively heating the article with a light source, to a temperature at which the article is fluent; molding the article into a desired conformation; and modifying the state of the article to become non-fluent in the desired conformation.

In one embodiment, Roth, et al. provides an apparatus comprising a balloon dilatation catheter having an associated optical tip assembly. In operation, the polymeric material would be positioned on the balloon, initially in the form of a tube or a sleeve. An optical tip assembly directs light to the polymeric material. Upon absorption of the light, the polymeric material is heated to a temperature at which it becomes moldable. Inflation of the balloon, then, causes the moldable polymeric material to expand outwardly, thereby pressing the heated polymer into contact with the tissue lumen.

Others have also proposed apparatuses and systems for local application of an energy source to polymerize an initially fluent material in vivo. For example, U.S. Pat. No. 6,468,520 to Rowe, et al., which is incorporated herein by reference, provides devices for applying a material to a surface of targeted tissue within a patient and polymerizing the material in vivo. Typically, the coating on the tissue is applied as a predetermined volume of prepolymer composition. After application, the coating is irradiated with light from an applicator to initiate polymerization.

The Rowe, et al. applicator is designed for use in open surgery procedures and in a laparoscopic environment. The embodied applicator includes a handle and a hollow, rigid shaft, which is attached to and extends distally from the handle. The distal tip of the shaft includes an emission nozzle, which is disposed in the hollow cavity of the rigid shaft, from which the prepolymer liquid is sprayed and atomized by compressed gas. Also disposed at the distal end of the shaft is the emission aperture of an optical path, which also is disposed in the hollow cavity of the rigid shaft, that is structured and arranged to irradiate the sprayed tissue with light.

More specifically, the distal end of the shaft of the applicator is positioned approximately two (2) centimeters from the surface of tissue to be treated. The applicator includes an emission nozzle that applies a thin film of sealant to a portion of the soft tissue. The applicator also includes an optical fiber or a bundle of such fibers to transmit, or couple, light from a light source to the light emission aperture. A microprocessor controls the light source by switching the light source on and off. The light is switched on after the pumping stroke of the emission nozzle has been completed and is allowed to remain on for a predetermined length of time sufficient to assure full polymerization of the sprayed composition.

U.S. Pat. No. 6,611,110 to Fregoso discloses a light-weight, portable, battery-powered photocuring device having a single, high power LED as the light source. The Fregoso device, however, includes an expensive driving circuit comprising an inductive storage device, a switching regulator device, a rectifier, a filter, and a current sensing device. The driving circuit powers, i.e., “drives”, the LED. The inductive storage device permits driving the LED with minimal voltage. The switching regulator device protects the diode from burning out by monitoring and regulating the power, i.e., the current, applied to the LED. The rectifier and filter for converting alternating current (AC) to direct current (DC). The current sensing unit, comprising a current driver and a temperature compensation circuit, and, optionally, a phototransistor, controls the current supplied to the LED to protect the LED from adverse temperature effects and to drive the LED at or near its maximum current.

However, there are problems with the prior art devices. First, they tend to be bulky and, second, they tend to be relatively expensive, requiring hospitals and clinics to make serious capital outlays. Accordingly, it would be desirable to provide a relatively inexpensive, hand-held, single-use, disposable photo-polymerization device for polymerizing light-sensitive sealants for a variety of surgical applications.

### SUMMARY OF THE INVENTION

In one embodiment, the present invention provides a self-contained, hand-held, disposable device for providing activating energy to a site of application of a light-sensitive material, e.g., a fluid sealant, hydrogel or a polymer, on mammalian tissue. When the light-sensitive material has been adequately illuminated or irradiated, it cures, polymerizes, bonds, and/or cross-links with the mammalian tissue. Preferably, the device comprises a light source, e.g., one or more low-power, light emitting diodes, laser diodes, halogen lamps, metal halide lamps, and the like, that emits a desired power density, or intensity, of light for a predetermined period of time, to illuminate or irradiate the site of application of the light-sensitive material; a power source for providing current to drive the light source; and a control board for controlling the predetermined period of time of the light source and the magnitude of the current supplied to the light source. Moreover, the control board controls current to the light source to ensure that current above a threshold current is provided. The threshold current is set at a specific limit to ensure that the light source is sufficiently bright to cure, polymerize, bond and/or cross-link the polymer material in the designated amount of time.

In one aspect of the first embodiment, the control board is hardwired or includes a microprocessor to control the light source duty cycle to between about 40 seconds and about 60 seconds ON. More preferably, the control board controls the light source duty cycle to about 50 seconds ON.

In another aspect of the first embodiment, the control board overdrives a relatively low-power light source that otherwise could not provide sufficient output power, i.e., power density or light intensity, by providing more current to the light source than the light source is rated for. More preferably, the control board overdrives the light source by providing several times as much current to the light source as the light source is rated for. This allows for use of less expensive, lower power diodes.

Preferably, the control board is hardwired to provide a current limiting system that controls the voltage and turn on time of the light source, to ensure that the light source provides adequate light intensity for a predetermined amount of time. Alternatively, the control board can include a microprocessor having sensors to monitor the power density, i.e., light intensity, emitted by the light source, which modifies the amount of current provided to the light source and/or the time of application of the light based on the monitored power density of the light source.

The device further optionally comprises one or more collimating lenses for focusing light incident thereon; and an optical shaft, e.g., a flexible or rigid optical fiber or a bundle of optical fibers, wherein the one or more collimating lenses focuses incident light from the light source onto a proximal end of the optical shaft, which is further transmitted and emitted from a tip at the distal end of the optical shaft; and wherein the emitted light illuminates or irradiates the site of application of the light-sensitive material to cause the light-sensitive material to cure, polymerize, and/or cross-link with the mammalian tissue. Alternatively, the device further optionally comprises a flexible shaft having a light source structured and arranged at a distal end thereof.

Preferably, the desired power density of light for illuminating or irradiating the light-sensitive material is selected based on the requirements for the sealant, hydrogel, and the like used. Indeed, different sealant types cure at different wavelengths or wavelength bands. For example, for a FocalSeal L sealant, the desired power density can be greater than or equal to about 15 mW/cm² and less than about 100 mW/cm² and the desired emitted light is within the visible light spectrum. More preferably, for FocalSeal L sealant, the emitted light has a wavelength between about 420 nm and about 560 nm. Nor is the present invention limited to the visible light spectrum. For example, ultra-violet with a wavelength range between about 365 nm and about 405 nm can be used without deviating from the scope and spirit of this disclosure.

In another embodiment, the present invention comprises a system for providing activating energy to a site of application of a light-sensitive material on mammalian tissue, the system comprising:

a hand-held, disposable device having
a light source, which emits a desired power density of light for a predetermined period of time, that illuminates or irradiates the site of application of the light-sensitive material;
a power source for providing current to drive the light source; and
a control board for controlling the predetermined period of time of said light source,
wherein the control board controls current to the light source to ensure that current above a threshold current is provided; and

a cannula, a trocar, or a combination thereof for introducing the hand-held device into a mammalian patient;

wherein the light-sensitive material is applied to a desired location on the mammalian tissue through the cannula, trocar, or combination thereof and the discrete location is illuminated or irradiated by light emitted by the device to cure, polymerize, and/or cross-link the light-sensitive material to the mammalian tissue at the discrete location.

In yet another embodiment, the present invention provides a method for treating a discrete location on mammalian tissue. Specifically, the method comprises the steps of:

applying a light-sensitive material to the discrete location;

illuminating or irradiating the light-sensitive material using a device that illuminates or irradiates the light-sensitive material with a desired power density of light for a predetermined period of time; and controlling the desired power density of light and the period of time

wherein the desired power density is controlled by providing current to the device that above a threshold current.

Preferably, the step of illuminating the light-sensitive material includes the sub-steps of positioning a distal end of the device a short distance, e.g., about two (2) centimeters, from the discrete location. Advantageously, if a relatively low-power, inexpensive light source is used, the further includes the sub-step of overdriving the light source by providing more current to the light source than the light source is rated for, e.g., several times as much current.

Optionally, when a device having an optical shaft is used, the step of illuminating the light-sensitive material further includes the sub-steps of:

emitting light from the light source, e.g., a low-power LED, laser diode, halogen lamp, metal halide lamp, and the like, on one or more collimating lenses;

focusing incident light on the one or more collimating lenses on a proximal end of the optical shaft;

transmitting the incident light on the proximal end of the optical shaft to a distal end of said optical shaft; and

emitting light from the distal end of the optical shaft, e.g., at a desired light power density, for a predetermined period of time, to illuminate or irradiate the discrete location to cause the light-sensitive material to cure, polymerize, or cross-link with the mammalian tissue.

Preferably, in the embodied methods, the step of illuminating or irradiating the light-sensitive material for a predetermined period of time includes illuminating or irradiating the light-sensitive material for a duty cycle that consists of between about 40 seconds and about 60 seconds ON and more preferably, the step includes illuminating or irradiating the light-sensitive material for a duty cycle that consists of about 50 seconds ON.

In one aspect of the embodied methods, step of controlling the desired power density of light and the period of time includes the sub-steps of monitoring the power density emitted by the light source and modifying the duty cycle time of application and/or modifying the current provided to the light source. Preferably, the step of controlling the desired power density of light includes hardwiring the device so to control the duty cycle and the voltage delivered to the light source. Alternatively, the step of controlling the desired power density includes providing a microprocessor to control the duty cycle and current flow.

### BRIEF DESCRIPTION OF THE DRAWINGS

The invention will be better understood by reference to the following more detailed description and accompanying drawings where like reference numbers refer to like parts:

FIG. 1 is an illustrative embodiment of a hand held, single use, disposable LED device for curing, polymerizing, and/or cross-linking a light-sensitive material applied to mammalian tissue;

FIG. 2 is a graph showing the relationship between the sealant strength (“modulus”) for FocalSeal L and the time of application of light for three light power densities;

FIG. 3 is a dual graph showing the relationship between drive current and power density and the relationship between drive current and sealant modulus of a fresh and a degraded sealant sample;

FIG. 4 is a graph showing the relationship between time of application of light versus temperature for a 50 seconds ON and 30 seconds OFF duty cycle;

FIG. 5 is a diagrammatic of the optics for light transfer from the LED to the distal end of the rigid optical fiber; and

FIG. 6 is a flow chart of an embodied method of treating a discrete location of mammalian tissue in accordance with the present invention; and

FIG. 7 is another illustrative embodiment of a hand held, single use, disposable LED device for curing, polymerizing, bond, and/or cross-linking a light-sensitive material applied to mammalian tissue.

### DETAILED DESCRIPTION OF THE PREFERRED EMBODIMENTS

In its broadest terms, the present invention discloses self-contained, low-cost, disposable devices for providing activating energy, e.g., visible light, for the photopolymerization of light-sensitive materials, e.g., surgical sealants, hydrogels, and the like, used in surgical applications and systems and methods using such devices. In one embodiment of the present invention, a light-emitting diode (“LED”) is structured and arranged on the distal end of a flexible or rigid shaft of an applicator and in another embodiment, an LED is structured and arranged at the proximal end of a flexible or rigid optical shaft of an applicator. The former embodiment provides enhanced optical attributes; however, heat dissipation is a greater concern, whereas, the latter embodiment provides better thermal maintenance; however, optical efficiency suffers.

The current inventors have studied sealant strength, or sealant “modulus” extensively, to evaluate its effect on polymer-tissue cross-linking efficiency. The independent variables of the study include power density, wavelength of the light source, and time of application. As a point of departure, the inventors noted that current specifications for acceptable cross-linking of a FocalSeal-L surgical sealant (previously manufactured by Focal, Incorporated of Lexington, Mass. but now manufactured by Genzyme, Incorporated of Cambridge, Mass.) require a minimum power density of about 100 mW/cm², a light source wavelength range between about 470 nm to about 520 nm, and a time of application of about 40 seconds.

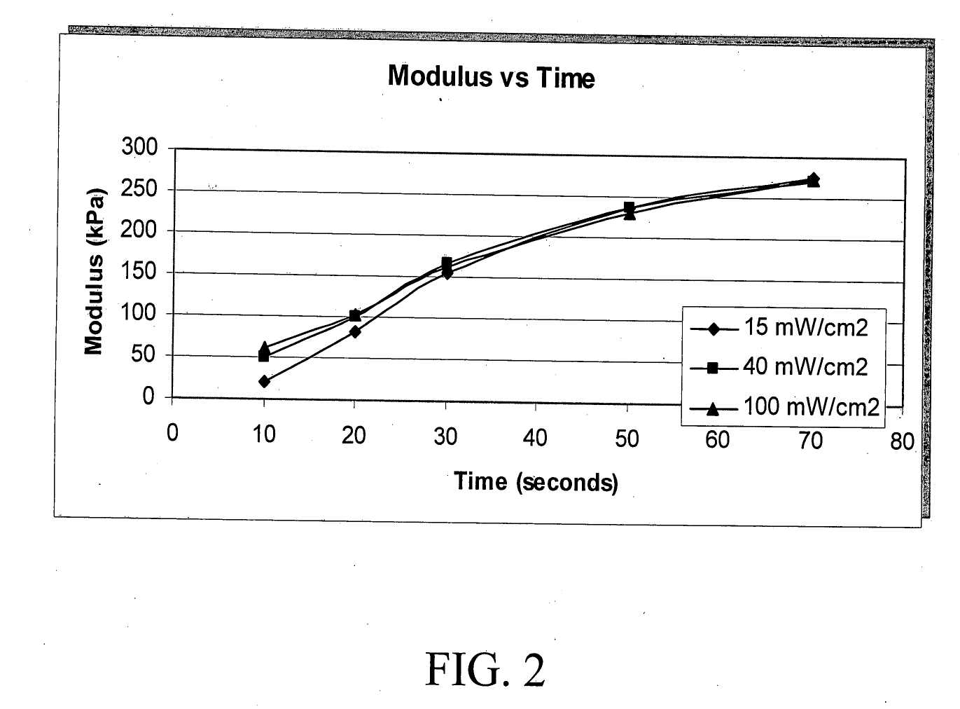

Referring to FIG. 2, the inventors observed that, for any time of application greater than about 40 seconds, the modulus is virtually unaffected by power density. Indeed, the inventors discovered that, for a FocalSeal L sealant, the cross-linking reaction saturates at a power density of about 15 mW/cm². Accordingly, additional light energy beyond 15 mW/cm² does not provide further cross-linking. In short, at a time of application of about 40 seconds, a power density of only about 15 mW/cm² is required to irradiate the sealant material, rather than a power density of about 100 mW/cm².

Those skilled in the art recognize that, power density is a function of the intensity of the light and the size of the spot. Moreover, because the cost of a light source is often directly proportional to its power output, a practical way of maximizing power density is to provide a tiny focus area. This variable, however, is a function of the numerical aperture and the distance from the light source to the targeted area, which can be controlled in choosing a light source and in positioning the light source with respect to the targeted area.

Light sources that can provide a relatively high power density are relatively bulky and expensive, neither of which lends itself to an inexpensive and/or disposable light source. In contrast, relatively low power LEDs are abundantly available and relatively inexpensive, which attributes are conducive to an inexpensive, disposable light source.

With further research of LEDs as an activating, i.e., illuminating or irradiating, light source, the inventors have also discovered that conventional practice rates, or categorizes, LEDs photometrically, which relates to how a human eye perceives light intensity. Indeed, LEDs are customarily binned based on their photometric output in lumens per Watt (“Lm/W”), their light color in nanometers (“nm”), and their forward bias voltage. For example, a LumiLED Bin Q LED, which is manufactured by LumiLED of Sunnyvale, Calif., is rated at 30.6 to 39.8 lumens per Watt (“Lm/W”). For a wavelength of about 490 nm, the LumiLED provides power of about 163 mW/W to about 213 mW/W and for a wavelength of about 515 nm, the LumiLED provides power of about 78 mW/W to about 102 mW/W.

However, the number of photons, which is to say the radiometric capability of the LED, and not the photometric capability of the LED affects cross-linking efficiency. Thus, for the same LumiLED Bin Q LED, the power density for different wavelengths can vary between about 30 and about 50 percent, which is not acceptable when saturation results at a power density of about 15 mW/cm² for a FocalSeal L sealant. Accordingly, power control of LEDs is required until LEDs are also binned based on their power density. Recent developments in the dental field have led to limited radiometric binning of LEDs, which will better control the variability of power requirements.

In testing their theoretical approach to the design problem, the inventors tested some relatively inexpensive, readily available LEDs for their ability to cure “fresh” commercial sealant, e.g., FocalSeal-L surgical sealant, and degraded sealant by photopolymerization. Surgical sealant such as FocalSeal-L is a synthetic, liquid sealant that can be applied to mammalian tissue, e.g., the lungs, the epidermis, etc., that is photo-curable when illuminated or irradiated by a light source. Degraded sealant corresponds to sealants that have degraded in strength, e.g., whose shelf life has expired. The inventors, further, also analyzed the effect of “overdriving” a light source on sealant modulus and power density. Overdriving refers to a technique for driving an LED by providing significantly more current to the LED than the rated driving current.

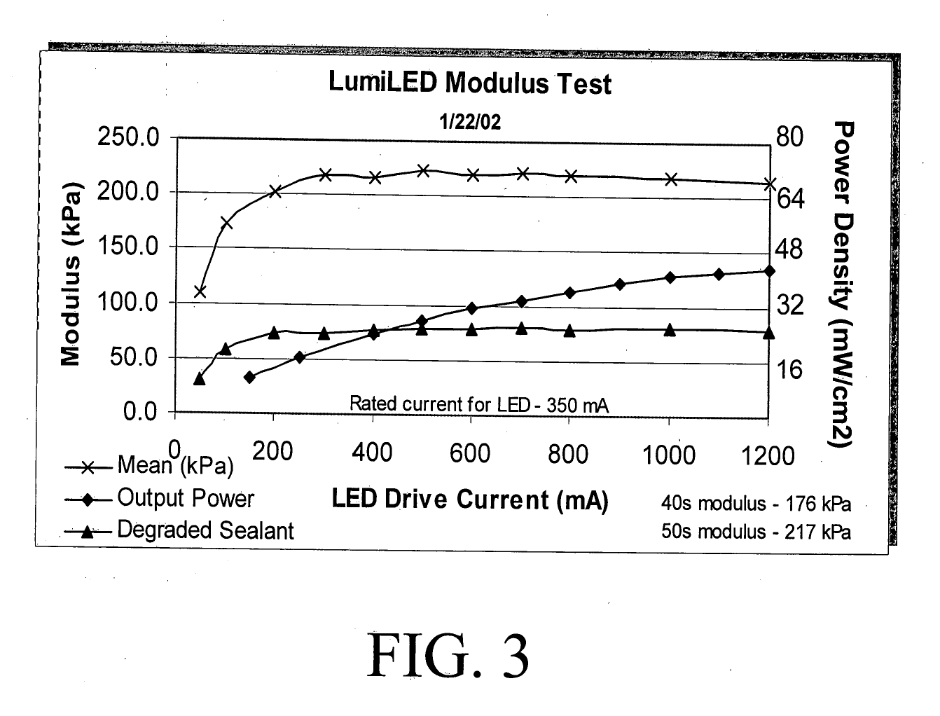

As shown in FIG. 3, an LED with a rated driving current of about 350 mA, can provide a power density of about 20 mW/cm² for a 50 second cure time.

Moreover, when the LED is overdriven instead at about 1200 mA, a power density of about 45 mW/cm² is produced. As a result, a significant, e.g., a 2.25 times, increase in power density can be achieved simply by overdriving the LED. As a result, less powerful—and therefore less expensive—LEDs can be used and overdriven to a higher power density.

FIG. 3 shows that overdriving LEDs does not adversely impact the modulus of a commercial sealant, which is about 220 kPa between the rated drive current of about 350 mA and 1200 mA, or the modulus of a degraded sealant, which is about 75 kPa between the rated drive current of about 350 mA and 1200 mA. Accordingly, because the preferred use of the embodied invention assumes that the disposable LED light source will be used only once and then discarded, the long- and short-term effects of overdriving the LED are moot. The advantages of not affecting the modulus and providing a significantly higher, i.e., 2.25 times, power density make it possible to use an even lower power LED.

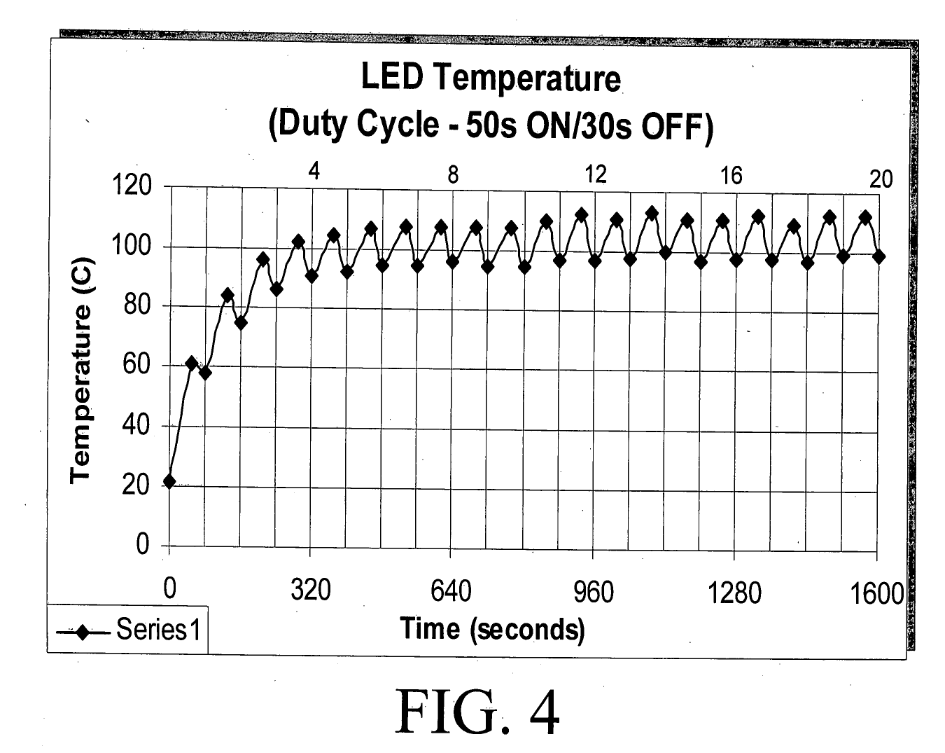

Overdriving an LED for about 50 seconds, however, can generate excess heat that can damage the device, e.g., melt junction leads and the like, or, possibly, be deleterious to other tissues or fluids in the vicinity of the light. As a result, through steady state thermal resistance modeling, the inventors have discovered that a heat sink can effectively maintain the temperature around a junction lead below about 300 degrees Centigrade (° C.). Moreover, referring to FIG. 4, temperature cycle analyses including overdriving an LED at 1250 A repeatedly for a duty cycle of 50 seconds ON and 30 seconds OFF demonstrated no significant loss in light output.

### Embodiment 1

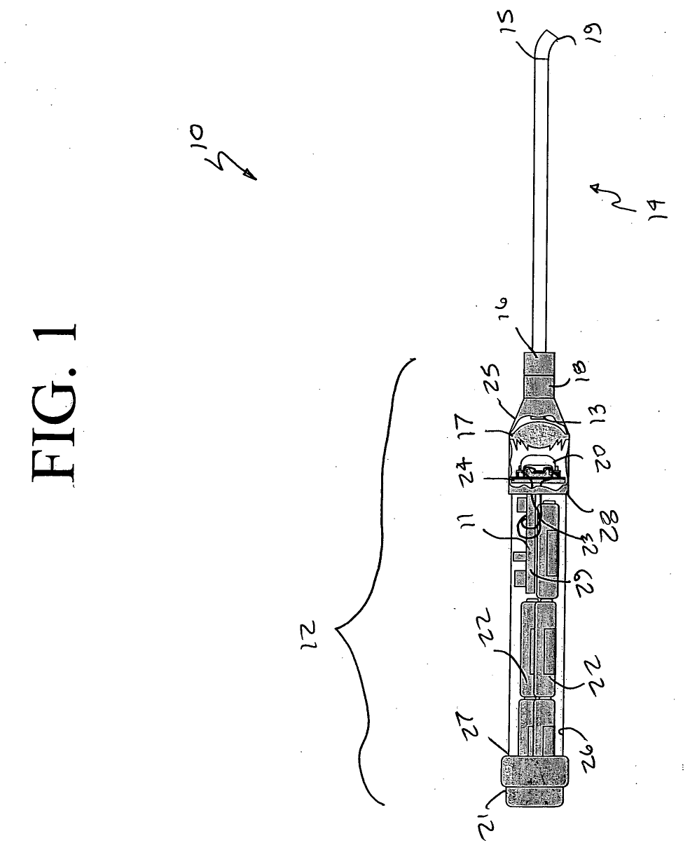

Having described the inventors' research, we will now describe a first embodiment of a low cost, self-contained, hand-held, single-use, disposable, LED device that provides activating energy to cure, polymerize, bond, and/or cross-link a light-sensitive polymerization initiator, i.e., a “sealant material”, that has been applied to mammalian tissue and the like. Referring to FIG. 1, an illustrative embodiment of a self-contained, hand-held sealant-activating device 10 is shown. In the context of this first embodiment, the sealant-activating device 10 will be referred to as an LED device 10 and its light source 20 will be referred to as an LED 20. This convention is not to be construed as limiting the invention as claimed to LED light sources. On the contrary, other light sources, e.g., a laser diode, a halogen lamp, a metal halide lamp, and the like, can be used without violating the scope and spirit of this disclosure.

In a preferred embodiment, the embodied LED device 10 comprises a handle portion 12 and an optical shaft 14 that can be rigid or flexible. The handle portion 12 internally contains the works of the device 10. The optical shaft 14 transmits light to a tip 19 at its distal end 15. The handle portion 12 includes a removable bottom cap 21, a hollow or substantially hollow cylindrical shaft portion 26, and a top, housing portion 25. The bottom cap 21 and the top, housing portion 25 are removably attachable to the cylindrical shaft portion 26, respectively, at a proximal end 27 and a distal end 28. Among other features, the bottom cap 21 and the top, housing portion 25 provide access to the inner cavity of the cylindrical shaft portion 25.

In one aspect of the present invention, the bottom cap 21 and top, housing portion 25 can be removably attached to the cylindrical shaft portion 26 by friction fit, e.g., using a clamshell with a button arrangement or a pipe clamp. Alternatively, the bottom cap 21 and top, housing portion 25 and the proximal and distal ends 27 and 28 of the cylindrical shaft portion 26 can include corresponding threadings (not shown) so that the bottom cap 21 can be removably attached to the proximal end 27 of the cylindrical shaft portion 26 and the top, housing portion 25 can be removably attached to the distal end 28 of the cylindrical shaft portion 26, e.g., by screwing the bottom cap 21 and the top, housing portion 25 onto the threadings on the proximal and distal ends 27 and 28 of the cylindrical shaft portion 26, respectively. Other means of attaching the cylindrical shaft portion 26 to the bottom cap 21 and the top, housing portion are well-known to those skilled in the art can be employed without violating the scope and spirit of this disclosure.

The cylindrical shaft portion 26 includes an inner cavity in which a power source 22 and a control board 11 are structured and arranged. Preferably, the power source 22 can provide sufficient power to the LED 20 and to the control board 11 to enable the device 10 to perform photopolymer bonding and/or cross-linking efficiently. For example, for a FocalSeal-L sealant, a power density in excess of 15 mW/cm² but less than 100 mW/cm², an application time between about 40 and about 60 seconds (preferably about 50 seconds), and a visible light wavelength to provide a blue-green light, i.e., about 460-560 nm, are preferred. More preferably, the power source 22 comprises a disposable or replaceable power source, e.g., one or more AA or AAA batteries that are disposed in an array. Although FIG. 1 depicts a power source 22 comprising five batteries in an array, the invention is not to be construed as being so limited. Indeed, more or fewer batteries can be included in the device 10 as long as they provide sufficient power for efficiently operating the LED 20 and the control board 11.

In a preferred embodiment, a plurality of integrated circuits, electronic circuits, and/or electronic devices are hardwired on a substrate 29 of the control board 11 to provide a desired application time between about 40 and about 60 seconds (preferably about 50 seconds), and also to modify the driving current to the light source 20. In operation, a user can turn on the light source 20 using an ON button and the hardwired control board 11 will deliver the desired driving current for the desired duty cycle then shut OFF automatically. This process can be repeated as necessary to cure, polymerize, bond, and/or cross-link the light-activated material. More specifically, the control board 11 will control the amount of current to the light source 20 to ensure that the level of current provided to the light source 20 never dips below a threshold current that is necessary to provide the desired output power density.

Although more expensive than hardwiring, the control board 11, instead, could comprises a microprocessor that includes a plurality of integrated circuits, electronic circuits, and/or electronic devices that are structured and arranged on a substrate 29. The integrated circuits, electronic circuits, and/or electronic devices can include, without limitation, a processing unit, a random access memory (“RAM”), and a read-only memory (“ROM”) to operate the device 10 efficiently. Specifically, the ROM of the control board 11 can include one or more applications that control the duty cycle of the LED 20 using a switching unit (not shown). Indeed, the microprocessor of the control board 11 can control the light source 20 by switching the light source 20 ON and OFF, thereby controlling the duty cycle or time of application of the light.

For example, when activated, e.g., using an ON button, the control board 11 can drive the LED 20 ON for a predetermined length of time sufficient to assure full polymerization of the light-sensitive material (about 50 seconds), and then turn the LED 20 OFF. By controlling the duty cycle thusly, as demonstrated in FIG. 4, the temperature generated by the LED 20 can be controlled to prevent damage from overheating. Advantageously, when activated, the control board 11 can cyclically drive the LED 20 ON for a predetermined length of time sufficient to assure full polymerization of the light-sensitive material (about 50 seconds), and then turn the LED 20 OFF for a predetermined length of time, then ON again, et cetera.

The microprocessor of the control board 11 controls the light source 20 also by controlling the amount of current flowing to the LED 20 when it is on. For example, the control board 11 can overdrive the LED 20 by providing several times more current to the LED 20 than the LED 20 is rated for. By controlling the current magnitude, as demonstrated in FIG. 3, the power density of the LED 20 can be increased many-fold. Furthermore, the microprocessor of the control board 11 will control the amount of current to the light source 20 to ensure that the level of current provided to the light source 20 never dips below a threshold current that is necessary to provide the desired output power density.

The ROM of the control board 11 can also include one or more applications designed, e.g., to monitor the operating temperature of the device 10 to make adjustments to the duty cycle; to control the amount of current being delivered to overdrive the LED 20; and/or to monitor and control the power density output of the LED 20, and the like.

Preferably, the top, housing portion 25 of the device 10 includes openings at both ends and an inner chamber therebetween. The larger end of the top, housing portion 25 is used to removably attach the top, housing portion 25 to the cylindrical shaft portion 26 and the smaller end of the top, housing portion 25 receives the proximal end 13 of the optical shaft 14.

In one embodiment, the optical shaft 14 is removably attachable to the top, housing portion 25 of the LED device 10. For example, the optical shaft 14 can be removably attached to the handle portion 12 by slip fit using a setscrew to hold the optical shaft 14 in place. Alternatively, the optical shaft 14 can be structured and arranged to fit through an opening in a first threaded portion 16 that can be rotated onto a second threaded portion 18 to provide a tight interference fit between the threadings of the first and second threaded portions 16 and 18. The tight interference fit between the first and second threaded portions 16 and 18 also provides a tight interference fit between the rigid optical shaft 14 and the top, housing portion 25.

Preferably, an LED 20 and a collimating lens 17 can be structured and arranged within the inner cavity of the top, housing portion 25. Although the LED 20 and the collimating lens 17 are referred to in the singular in this disclosure, the invention is not to be construed as being so limited. Indeed, a plurality of smaller LEDs 20 and/or multiple lenses 17 can be used without violating the scope and spirit of this disclosure.

In a preferred embodiment, the light source 20 is a low power LED 20 such as those manufactured by LumiLED of Sunnyvale, Calif. The LumiLED brand diodes are particularly suitable for this application because they are extremely bright, i.e., have a high intensity, and are relatively small. A pair of lead wires 23 provides current (power) to drive the LED 20. A switching unit (not shown), which is controlled by an application in the control board 11, controls the flow of current from the power source (batteries) 22 to the LED 20.

Overdriving the LED 20 using relatively lower power LEDs 20, e.g., a 1 W LED, and using relatively higher power LEDs 20, e.g., a 5 W LED, can generate significant amounts of heat that require dissipation or removal. In one aspect of the present invention, a heat sink 24 is structured and arranged at the back of the LED 20, between the LED 20 and the ambient or substantially ambient conditions in the inner cavity of the cylindrical shaft portion 26. Preferably, the heat sink 24 is manufactured of one or more relatively highly thermally conductive material, e.g., aluminum, carbon-carbon materials, copper, stainless steel, and the like. In most case, conventionally manufactured LumiLEDs 20 include integral heat sinks 24 Optionally heat removal can be effected by designing the second threaded portion 18 of the top, housing portion 25 as a heat sink.

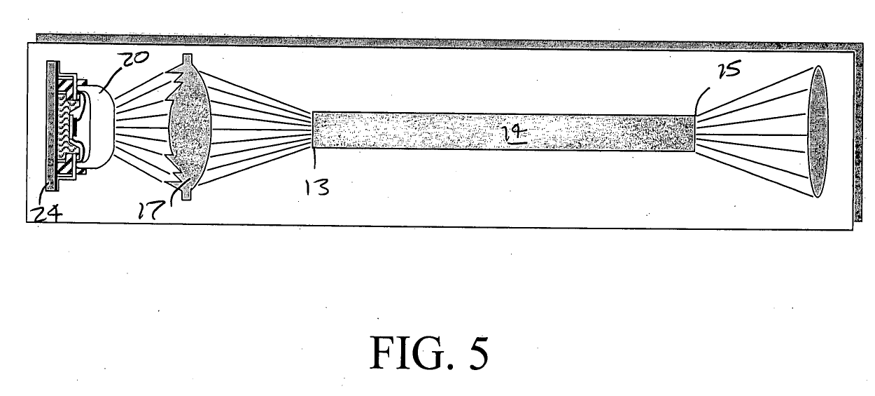

In a preferred embodiment, the optical shaft 14 includes an optical fiber, e.g., a glass fiber, or a bundle of such fibers to transmit, or couple, light from the LED 20 to the light emission aperture at the distal end 15 of the shaft 14. The optical shaft 14 can be flexible or rigid. Referring to FIG. 5, the optical shaft 14 has a proximal end 13 and a distal end 15. The proximal end 13 is disposed through the smaller opening in the top, housing portion 25 in the vicinity of a collimating lens 17. The distal end 15 of the optical shaft 14 includes a tip 19 that can be straight or slightly curved.

Referring to FIG. 5, in use, the distal end 15 of the rigid optical shaft 14 can be disposed perpendicular or substantially perpendicular to and, preferably, within a short distance, e.g., about two (2) centimeters, of an area onto which a light-sensitive polymerization initiator, i.e., a sealant material such as FocalSeal-L, has been applied, e.g., on a mammalian tissue. When the LED device 10 is turned ON, the control board 11 opens the switching element and current flows to the LED 20 for the duration of the duty cycle, causing the LED 20 to emit light. Preferably, during its duty cycle, the LED 20 emits visible light, e.g., blue-green light, that is incident on the collimating lens 17. The light incident on the collimating lens 17 is then focused on the proximal end 13 of the optical shaft 14. The light incident on the proximal end 13 of the optical shaft 14 travels to the distal end 15 and the tip 19. Light emitted from the tip 19 is emitted at sufficient power density to cure, polymerize, and/or cross-link the polymer sealant and the mammalian tissue efficiently, without damaging the nearby tissues or fluids.

The ability to selectively bond or cross-link an implanted polymeric material using light in the visible, near-visible, and/or ultra-violet spectrum can be achieved using a light source 20 that produces a wavelength spectrum that is not readily absorbed by body tissue. Preferably, light from the source is used to cross-link a polymeric material that is at least partially absorptive of the light in the spectral range. Light that is not absorbed by the sealant material can be absorbed by a relatively large area of tissue as it penetrates beyond the polymer. As such, resultant heating occurs throughout a much larger volume of tissue. Since the temperature rise in the tissue is a function of energy absorbed within a unit volume of tissue, localized heating is significantly lower as compared to the heating caused by wavelengths that are readily absorbed, i.e., by a smaller volume of tissue. The requirement for wavelengths that have low tissue absorption characteristics is necessary only to the extent that excess heating of the tissue does not occur or is undesirable at the particular treatment location.

Alternatively, it is possible to use light having a spectrum that is absorbed by body tissues and body fluids provided that the polymeric material is highly absorptive of light in the spectral range. In this case, the polymer will absorb substantially all of the light, thereby minimizing the amount that is transferred to the body tissue and minimizing the heating effect of that light on tissue.

### Embodiment 2

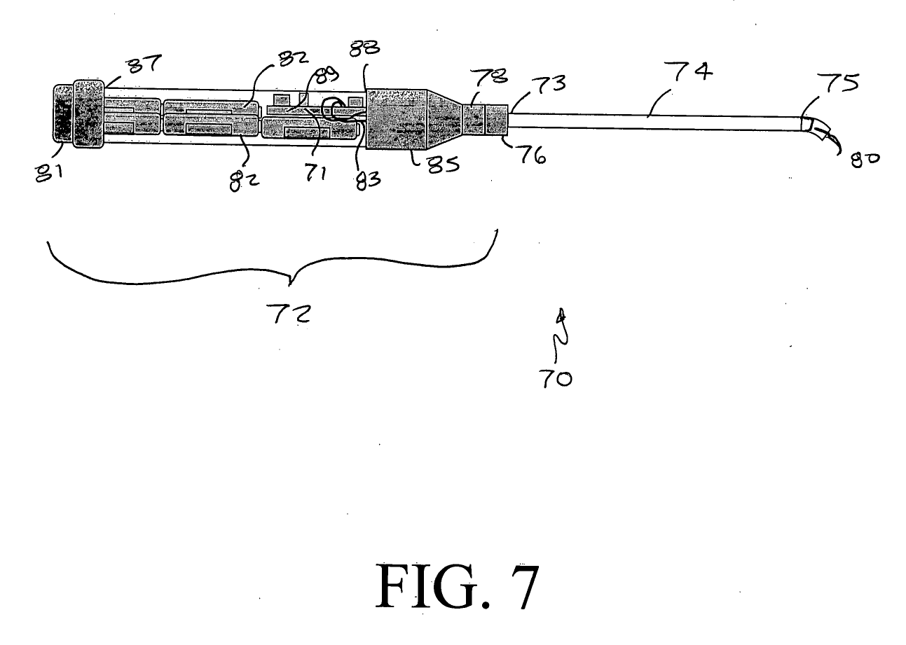

Having described a first embodiment of an LED device, we will now describe an alternate embodiment for a low cost, self-contained, hand-held, single-use, disposable, LED device that provides activating energy to cure, cross-link, bond, and/or photopolymerize a light-sensitive polymerization initiator, i.e., a “sealant material”, that has been applied to mammalian tissue and the like. Referring to FIG. 7, an illustrative embodiment of a hand-held sealant-activating device 70 is shown. As with the previous embodiment, the sealant activating light device 70 will be referred to as an LED device 70 and the light source 80 for the device 70 will be referred to as an LED 80. This convention, again, is not to be construed as limiting the invention as claimed to LED light sources. Other light sources, e.g., laser diodes, halogen lamps, metal halide lamps, and the like can be used without violating the scope and spirit of this disclosure.

In a preferred embodiment, the LED device 70 comprises a handle portion 72 and a relatively flexible shaft 74. The handle portion 72 includes a removable bottom cap 81, a hollow or substantially hollow cylindrical shaft portion 86, and a top portion 85. The bottom cap 81 and the top portion 85 are removably attachable to the cylindrical shaft portion 86, respectively, at a proximal end 87 and a distal end 88. Among other features, the bottom cap 81 and top portion 85 provide access to the inner cavity of the cylindrical shaft portion 86.

In one aspect of the present invention, the bottom cap 81 and top portion 8 can be removably attached to the cylindrical shaft portion 86 by friction fit, e.g., using a clam shell with a button arrangement or a pipe clamp. Alternatively, the bottom cap 81 and top portion 85 and the proximal and distal ends 87 and 88 of the cylindrical shaft portion 86 can include corresponding threadings so that the bottom cap 81 can be removably attached to the proximal end 27 of the cylindrical shaft portion 86 and the top portion 85 can be removably attached to the distal end 88 of the cylindrical shaft portion 26, e.g., by screwing the bottom cap 81 and the top portion 85 onto the threadings on the proximal and distal ends 87 and 88 of the cylindrical shaft portion 86, respectively.

The cylindrical shaft portion 86 includes an inner cavity in which a power source 82 and a control board 71 are structured and arranged. Preferably, the power source 82 can provide sufficient power to the LED 80 and to the control board 71 to perform photopolymer cross-linking efficiently. For example, for a FocalSeal-L sealant, a power density in excess of about 15 mW/cm² but less than 100 mW/cm², an application time between about 40 and about 60 seconds (preferably about 50 seconds), and a visible light wavelength to provide a blue-green light, i.e., about 460-560 nm, are preferred. More preferably, the power source 82 comprises a disposable or replaceable power source, e.g., one or more AA or AAA batteries that are disposed in an array. Although FIG. 7 depicts a power source 82 comprising five batteries in an array, the invention is not to be construed as being so limited. Indeed, more or fewer batteries can be included in the device 70 as long as they provide sufficient power for efficiently operating the LED 80 and the control board 71.

In a preferred embodiment, a plurality of integrated circuits, electronic circuits, and/or electronic devices are hardwired on a substrate 89 of the control board 71 to provide a desired application time between about 40 and about 60 seconds (preferably about 50 seconds), and also to modify the driving current to the light source 80. In operation, a user can turn on the light source using an ON button and the hardwired control board 71 will deliver the desired driving current for the desired duty cycle then shut OFF automatically. More specifically, the control board 71 will control the amount of current to the light source 80 to ensure that the level of current provided to the light source 80 never dips below a threshold current that is necessary to provide the desired output power density. This process can be repeated as necessary to cure, polymerize, and/or cross-link the light-activated material.

Although more expensive than hardwiring, the control board 71, instead, could comprise a microprocessor that includes a plurality of integrated circuits, electronic circuits, and/or electronic devices that are arranged on a substrate 89. The integrated circuits, electronic circuits, and/or electronic devices include, without limitation a processing unit, RAM, and ROM to operate the device 70 efficiently. Specifically, the ROM of the control board 71 can include one or more applications that control the duty cycle of the LED 80 using a switching unit (not shown). Indeed, the microprocessor of the control board 71 controls the light source 80 by switching the light source 80 ON and OFF. For example, when activated, e.g., using an ON button, the control board 71 can drive the LED 80 ON for a predetermined length of time sufficient to assure full polymerization of the light-sensitive material (about 50 seconds), and then turn the LED 80 OFF). By controlling the duty cycle thusly, as demonstrated in FIG. 4, the temperature generated by the LED 80 can by controlled to prevent damage from overheating. Advantageously, when activated, the control board 71 can cyclically drive the LED 80 ON for a predetermined length of time sufficient to assure full polymerization of the light-sensitive material (about 50 seconds), and then turn the LED 80 OFF for a predetermined length of time, then ON again, et cetera.

The microprocessor of the control board 71 controls the light source 20 also by controlling the amount of current flowing to the LED 80 when it is on. For example, the control board 71 can overdrive the LED 80 by providing several times more current to the LED 80 than the LED 80 is rated for. By controlling the current magnitude, as demonstrated in FIG. 3, the power density of the LED 80 can be increased many-fold. Furthermore, the control board 71 will control the amount of current to the light source 80 to ensure that the level of current provided to the light source 80 never dips below a threshold current that is necessary to provide the desired output power density.

The ROM of the control board 71 can also include one or more applications designed, e.g., to monitor the operating temperature of the device 70 to make adjustments to the duty cycle; to control the amount of current being delivered to overdrive the LED 80; and/or to monitor and control the power density output of the LED 80, and the like.

Preferably, the top portion 85 of the device 70 includes openings at both ends and a chamber therebetween. The larger end of the top portion 85 is used to removably attach the top portion 85 to the cylindrical shaft portion 86 and the smaller end of the top portion 85 receives the proximal end 73 of the flexible shaft 74.

In one embodiment, the flexible shaft 84 is removably attachable to the top portion 85 of the LED device 70. For example, the shaft 84 can be removably attached to the handle portion 72 by slip fit using a setscrew to hold the shaft 84 in place. Alternatively, the flexible shaft 74 is structured and arranged to fit through an opening in a first threaded portion 76 that can be rotated onto a second threaded portion 78 to provide a tight interference fit between the threadings of the first and second threaded portions 76 and 78. The tight interference fit between the first and second threaded portions 76 and 78 also provides a tight interference fit between the flexible shaft 74 and the top portion 75.

The flexible shaft 74 provides heat removal and provides a conduit for delivering power, e.g., by wire leads 83, to the LED 80 that is disposed at the tip of the distal end 75 of the flexible shaft 74. As a result the flexible shaft 74 can be hollow, with the wire leads 83 running in the cavity, or the flexible shaft 74 can be solid, with the wire leads 83 embedded in the material making up the flexible shaft 74 or in a material disposed in the flexible shaft 74 for that purpose.

Preferably, the light source 80 at the tip of the flexible shaft 74 is a low power LED 80 such as those manufactured by LumiLED of Sunnyvale, Calif. The LumiLED brand diodes are particularly suitable for this application because they are extremely bright, i.e., have a high intensity, and are relatively small. A switching unit (not shown), which is controlled by an application in the control board 71, controls the flow of current from the power source (batteries) 82 to the LED 80.

In one aspect of the present invention, the flexible shaft 74 can be used advantageously to remove heat from the LED 80. Specifically, the flexible shaft 74 can be manufactured of one or more relatively highly thermally conductive material, e.g., aluminum, carbon-carbon materials, copper, stainless steel, and the like. Because the LED 80 is disposed at the distal end 75 of the flexible shaft 74, much of the heat generated by the LED 80 would be absorbed by the ambient environment without adequate heat removal.

In use, the LED 80 at the distal end 75 of the device 70 can be disposed perpendicular or substantially perpendicular to and, preferably, within a short distance, e.g., about two (2) centimeters, of an area onto which a sealant material comprising a light-sensitive polymerization initiator has been applied, e.g., on a mammalian tissue. When the LED device 70 is turned ON, the control board 71 opens the switching element and current flows to the LED 80 for the duration of the duty cycle, causing the LED 80 to emit light. Preferably, during its duty cycle, the LED 80 emits visible light, e.g., blue-green light, that is incident directly on the light-sensitive material. Light emitted can be emitted at sufficient power density to cure, polymerize, bond, and/or cross-link the polymer sealant and the mammalian tissue efficiently, without damaging the nearby tissues or fluids.

Here again, the ability to selectively bond and/or cross-link an implanted polymeric material using light in the visible, near-visible, and/or ultra-violet spectrum can be achieved using a light source 80 that produces a wavelength spectrum that is not readily absorbed by body tissue. Preferably, light from the source 80 is used to cross-link a polymeric material that is at least partially absorptive of the light in the spectral range. Light that is not absorbed by the sealant material is absorbed by a relatively large area of tissue as it penetrates beyond the polymer. As such, resultant heating occurs throughout a much larger volume of tissue. Since the temperature rise in the tissue is a function of energy absorbed within a unit volume of tissue, localized heating is significantly lower as compared to the heating caused by wavelengths that are readily absorbed, i.e., by a smaller volume of tissue. The requirement for wavelengths that have low tissue absorption characteristics is necessary only to the extent that excess heating of the tissue does not occur or is undesirable at the particular treatment location.

Alternatively, it is possible to use light having a spectrum that is absorbed by body tissues and body fluids provided that the polymeric material is highly absorptive of light in the spectral range. In this case, the polymer will absorb substantially all of the light, thereby minimizing the amount that is transferred to the body tissue and minimizing the heating effect of that light on tissue.

Having described two embodiments of a photo-polymerizing LED device 10 and 70, we will now discuss several surgical systems using the same. For example, one system comprises either of the above described LED devices 10 and 70 in combination with a medical or surgical cannula (not shown), trocar (not shown), and/or combination of the two. Medical cannulae and trocars are used extensively in a myriad of medical and surgical applications, e.g., to introduce an object into a patient and/or to remove an object from a patient. Typically, one or more small, local incisions can be made in the patient through which the distal ends of one or more cannulae can be introduced. Alternatively, a trocar can be used in lieu of a small incision to pass through the epidermis of the patient. A separate cannula can then be inserted into the cavity of the trocar or the trocar can be used in a similar fashion as a cannula

Generally, there is one incision made for use as a working cannula and another incision through which an optical device can be inserted to locate and observe the working cannula and the instruments being used therein. Advantageously, a device for applying a light-sensitive material in a fluent, semi-fluent, or non-fluent state can be inserted into the annulus of the cannula or trocar through the proximal end of the same. Once the light-sensitive material has been applied at a targeted area, the device can be extracted.

A device 10 and 70 can then be inserted into cannula and/or trocar to illuminate or irradiate the applied, light-sensitive material to cure, polymerize, bond, and/or cross-link the light-sensitive material to the mammalian tissue. When the light-sensitive material is not fluent, the intensity of the light delivered by the light source 20 and 80 first renders the material fluent and the tip 19 of the device 10 and 70 can used to mold or shape the material in contact with a tissue lumen. In a fluent state, light from the device 10 and 70 cures, polymerizes, bonds, and/or cross-links the light-sensitive material to the surrounding tissue.

A plethora of surgical uses of such a system already exist. For example, sealants can be applied to living tissue to prevent post-operative adhesions; to protect tissue surfaces; to alter the tissue; to create or preserve lumens, channels or reservoirs for the passage or collection of fluids; to create matrices for the growth of tissue; to control undesirable tissue growth; to deliver therapeutic agents to a tissue surface; to join a tissue surface to other tissue(s) or an artificial implant; to isolate or protect tissue or lesions to enable or mediate healing; to mediate the rate of substances or energy passing into, out of, or through the tissue; for the local application of biologically active species, and for the controlled release of biologically active agents to achieve local and systemic effects. Sealants, hydrogels, and the like, further, can be used as temporary or long-term tissue adhesives or as materials for filling voids in biological materials. The materials and conditions of application are selected to enhance desirable properties such as good tissue adherence without adverse tissue reaction, non-toxicity, good biocompatibility, biodegradability when desired, and ease of application or handling.

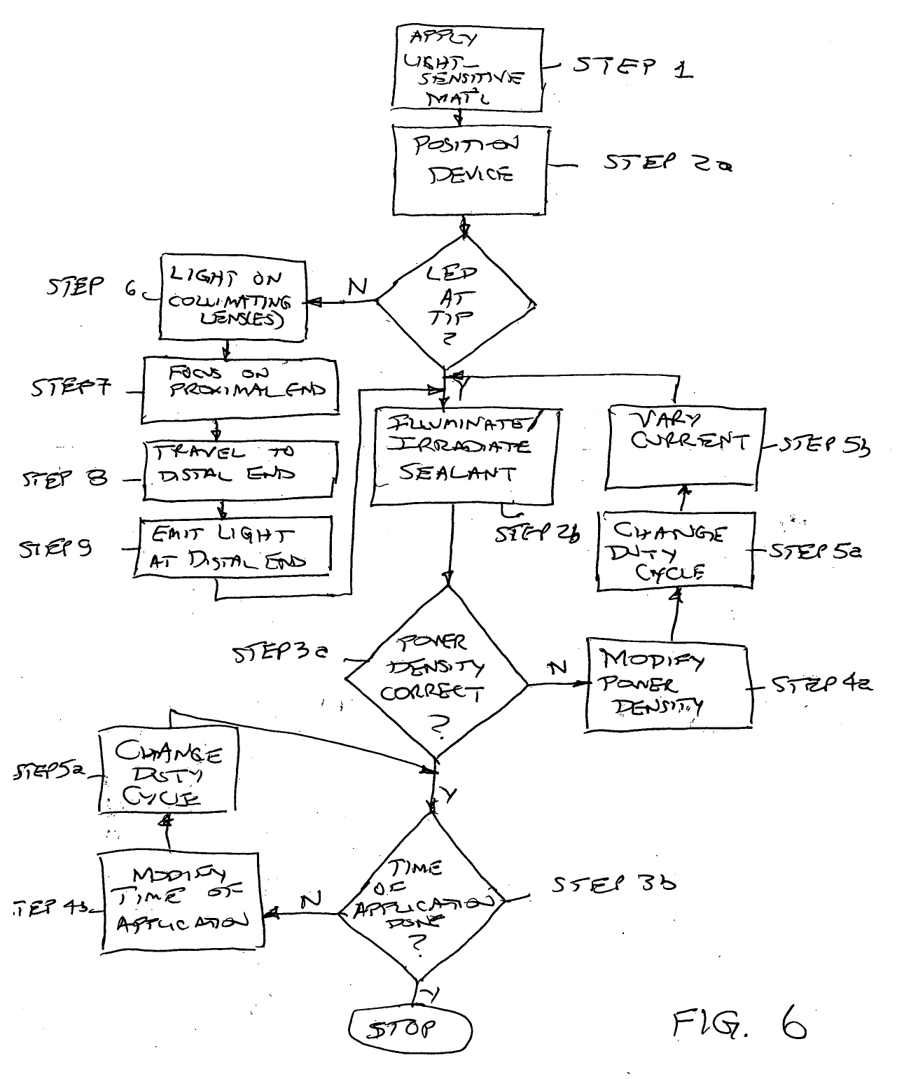

Having described various devices and systems for polymerizing light-sensitive sealants in vivo, we will now describe methods for treating a discrete, targeted site on mammalian tissue. Referring to FIG. 6, a flow chart of illustrative methods of treating a discrete location on mammalian tissue is shown. The embodied methods ensure that the light-sensitive material has sufficient sealant strength, i.e., sealant modulus, to accomplish its purpose.

The first step comprises applying a light-sensitive material to the discrete location (STEP 1). The prior art is replete with a myriad of methods of applying a thin film of a light-sensitive material, both fluent and non-fluent, to a discrete location in a mammalian patient laparoscopically or in an open surgical operation, all of which are included herein by reference, but none of which will be described in any great detail.

Once a thin film of light-sensitive material has been applied to the targeted area, a device for illuminating or irradiating the light-sensitive material such as any of the embodied devices previously described can be disposed in proximity of the discrete location (STEP 2a) and the discrete location can, further, be illuminated or irradiated by the light source (STEP 2b).

The dimensions of the targeted area, the numerical aperture of the light source, can affect the distance between the light source and the targeted area. For. example, preferably, the tip at the distal end of the device can be disposed a short distance from the discrete location. Generally, a distance of about two (2) centimeters with the shaft of the device oriented upright, i.e., perpendicular or substantially perpendicular, is desirable and practical. Such an arrangement can produce a focal spot of about 2 cm diameter, which corresponds to an area of about 3 cm². Thus, for a FocalSeal-L sealant, a power output of 45 mW would be required to provide a power density of 15 mW/cm². Those skilled in the art will realize that, when the illuminating or irradiating tip is closer than about two (2) centimeters, the focal area is smaller by the square of the radius of the illuminating or irradiating beam of light, hence, to maintain the power density of the light at the desired level, the power, or intensity, would have to decrease to compensate for the nearness. The opposite is also true. When the tip is further than about two (2) centimeters from the discrete location, the coverage area is larger by the square of the radius of the illuminating or irradiating beam of light, hence, to maintain the power density at the desired level, the power, or intensity, would have to increase to compensate for the greater distance.

Similarly, although the tip of the device can approach the discrete location from practically any angle, the efficiency of the illumination and irradiation of the light-sensitive material can be impaired and the distribution of the light can vary considerably between the portions of the discrete location that are closest to and the portions that are furthest from the tip of the device. For example, when the tip of the device is at an oblique to the targeted area, only the light-sensitive material closest to the light source may cure during the duty cycle and the light-sensitive material at the farthest point from the light source may not have been ideally cured. Additionally, with some sealants, hydrogels, and the like, the power density at the closest point to the oblique device could “overcure” the local area while the intensity and power density at the farthest point may “undercure” the local area. Here again, those skilled in the art will recognize that compensation for illuminating or irradiating the light-sensitive material when the device is not oriented perpendicular or substantially perpendicular to the discrete location angle may require different power densities and or times of application for different spots on the discrete location.

In a preferably embodiment, the light source, e.g., a low power light emitting diode, a laser diode, halogen lamp, metal halide lamp, and the like, is structured and arranged to provided a desired power density of light (STEP 3a) for a predetermined period of time (STEP 3b) to the light-sensitive material. The power density and predetermined period of time of application are designed to cure, polymerize, and/or cross-link the light-sensitive material so that the material and cross-linked tissue have a suitable strength, or modulus.

When the device is hardwired, the embodiment method includes the step of driving the light source by providing current to the same. Alternatively, when the device includes a microprocessor, the embodied method further includes the steps of monitoring and controlling, i.e., modifying, the desired power density of light (STEP 4a) and the period of time of application (STEP 4b). More specifically, the control board is structured and arranged to monitor and control the light source so that the light source illuminates or irradiates the light-sensitive material with a desired light power density (STEP 4a); and, moreover, the light source illuminates or irradiates the light-sensitive material for a duty cycle that consists of between about 40 seconds and about 60 seconds ON (STEP 4b). More preferably, the control board is structured and arranged to control the light source so that the light source illuminates or irradiates the light-sensitive material for a duty cycle comprising about 50 seconds ON.

The control board with a microprocessor can monitor the intensity of the light source (STEP 4a). In order to ensure that the light-sensitive material is exposed to a suitable amount of light, the control board can either modifying the duty cycle by varying the ON time with respect to the OFF time (STEP 5a) and/or by varying the amount of current provided to the light source (STEP 5b). As shown in FIG. 3, as the light source is overdriven by providing more current to the light source than the light source is rated for, the power density can be increased many times without having a deleterious effect on the sealant strength, or modulus.

In another embodiment, wherein the light source is not located at the tip of the device, but, rather, at the proximal end of an optical shaft, the step of illuminating the light-sensitive material includes directing light emitted from the light source on one or more collimating lenses (STEP 6); focusing incident light on the one or more collimating lenses on the proximal end of the optical shaft (STEP 7); transmitting the incident light on the proximal end of the optical shaft to the distal end of the optical shaft (STEP 8); and emitting light from the distal end of said optical shaft to illuminate or irradiate the discrete location to cause the light-sensitive material to cure, polymerize, and/or cross-link with the mammalian tissue (STEP 9).

Although preferred embodiments of the invention have been described using specific terms, such descriptions are for illustrative purposes only, and it is to be understood that changes and variations may be made without departing from the spirit or scope of the following claims.

For example, although in a preferred embodiment the device described herein is hand held and disposable, it would be possible to include a replaceable shaft that can be fitted on the handle portion quickly and securely. The replaceable shaft can then be disposable or sterilizable, e.g., using an autoclave.

What is claimed is:

1. A hand-held device comprising:
a light source for providing activating energy to an area of application of a light-sensitive material on mammalian tissue, the device,
wherein the light source emits a desired power density of light for a predetermined period of time that illuminates or irradiates the area of application of the light-sensitive material;
a power source for providing current to drive the light source; and
a control board for controlling the predetermined period of time of said light source,
wherein the control board controls current to the light source to ensure that current above a threshold current is provided.

2. The device as recited in claim 1, wherein the light source is one or more light emitting diodes.

3. The device as recited in claim 1, wherein the device is self-contained.

4. The device as recited in claim 1, wherein the light source is one or more laser diode, halogen lamps or metal halide lamps.

5. The device as recited in claim 1, wherein the power source comprises one or more batteries.

6. The device as recited in claim 1, wherein the predetermined period of time comprises a light source duty cycle that consists of between about 40 seconds and 60 seconds ON.

7. The device as recited in claim 6, wherein the light source duty cycle consists of about 50 seconds ON.

8. The device as recited in claim 1, wherein the control board is structured and arranged to overdrive the light source by providing more current to the light source than said light source is rated for.

9. The device as recited in claim 1, wherein the device is disposable.

10. The device as recited in claim 1, wherein the control board monitors the power density emitted by the light source.

11. The device as recited in claim 10, wherein the control board modifies the amount of current provided to the light source based on the monitored power density of the light source.

12. The device as recited in claim 1, wherein the light-sensitive material is a polymer that, when illuminated or irradiated, cures, polymerizes, bonds, and/or cross-links with the mammalian tissue.

13. The device as recited in claim 1, wherein the light-sensitive material is a fluid that, when illuminated or irradiated, cures, polymerizes, bonds, and/or cross-links with the mammalian tissue.

14. The device as recited in claim 1, wherein the device further comprises:
one or more collimating lenses for focusing light incident thereon; and
an optical shaft,
wherein the one or more collimating lenses focuses incident light from the light source onto a proximal end of the optical shaft, which is further transmitted and emitted from a tip at a distal end of said optical shaft; and
wherein the emitted light illuminates or irradiates the site of application of the light-sensitive material to cause said light-sensitive material to cure, polymerize or cross-link with the mammalian tissue.

15. The device as recited in claim 14, wherein the optical shaft is a rigid optical fiber or a bundle of optical fibers.

16. The device as recited in claim 14, wherein the tip is substantially straight or curved.

17. The device as recited in claim 14, wherein the light source, one or more collimating lenses, and proximal end of the optical shaft are structured and arranged within a housing assembly so that light emitted from the light source can travel to the tip of the optical shaft.

18. The device as recited in claim 17, wherein the optical shaft is removably attachable to the housing assembly.

19. The device as recited in claim 1, wherein the emitted light is within the visible light spectrum, the near visible light spectrum or the ultra-violet light spectrum.

20. The device as recited in claim 1, wherein the device further comprises a shaft having a proximal and a distal end;
wherein the light source is structured and arranged at the distal end of the shaft.

21. The device as recited in claim 20, wherein the shaft is manufactured of a material and structured and arranged to remove heat produced by the light source.

22. A system comprising:
a hand-held device having
a light source for providing activating energy to a targeted area of application of a light-sensitive material on mammalian tissue,
wherein the light source emits a desired power density of light for a predetermined period of time that illuminates or irradiates the targeted area of application of the light-sensitive material;
a power source for providing current to drive the light source; and
a control board for controlling the predetermined period of time and driving current of said light source
wherein the control board controls current to the light source to ensure that current above a threshold current is provided; and
a cannula for introducing the hand-held device into a mammalian patient;
wherein the light-sensitive material is applied to a desired location on the mammalian tissue through the cannula and the discrete location is illuminated or irradiated by light emitted by the device to cure, polymerize, bond, and/or cross-link the light-sensitive material to the mammalian tissue at said discrete location.

23. The system as recited in claim 22, wherein the system further includes a trocar for piercing a portion of the mammalian patient, wherein the cannula is structured and arranged to fit through the trocar.

24. The system as recited in claim 22, wherein the cannula is also a trocar.

25. A method for treating a discrete location on mammalian tissue, the method comprising the steps of:
applying a light-sensitive material to the discrete location;
illuminating or irradiating the light-sensitive material using a device as recited in claim 1, wherein the device illuminates or irradiates said light-sensitive material with a desired power density of light for a predetermined period of time; and
controlling the desired power density of light and the period of time,
wherein the desired power density is controlled by providing current to the device that is above a threshold current.

26. The method as recited in claim 25, wherein the step of controlling the desired power density of light includes overdriving the light source of the device by providing more current to said light source than said light source is rated for.

27. The method as recited in claim 25, wherein the step of illuminating the light-sensitive material includes the sub-steps:
emitting light from the light source on one or more collimating lenses;
focusing incident light on the one or more collimating lenses on a proximal end of an optical shaft;
transmitting the incident light on the proximal end of the optical shaft to a distal end of said optical shaft; and
emitting light from the distal end of said optical shaft to illuminate or irradiate the discrete location to cause the light-sensitive material to cure, polymerize, bond, or cross-link with the mammalian tissue.

28. The method as recited in claim 25, wherein the step of illuminating or irradiating the light-sensitive material includes illuminating or irradiating the material with one or more low-power light emitting diodes.

29. The method as recited in claim 25, wherein the step of illuminating or irradiating the light-sensitive material for a predetermined period of time includes illuminating or irradiating said light-sensitive material over the course of a duty cycle that consists of between about 40 seconds and 60 seconds ON.

30. The method as recited in claim 29, wherein the step of illuminating or irradiating the light-sensitive material for a predetermined period of time includes illuminating or irradiating said light-sensitive material over the course of a duty cycle that consists of about 50 seconds ON.

31. The method as recited in claim 37, wherein the step of controlling the desired power density of light includes the sub-steps of
monitoring the power density emitted by the light source; and
modifying the duty cycle.

32. The method as recited in claim 37, wherein the step of controlling the desired power density of light includes the sub-steps of
monitoring the power density emitted by the light source; and
modifying an amount of current provided to the light source.
